(19) United States
(12) Patent Application Publication
(10) Pub. No.: US 2023/0165940 A1
(43) Pub. Date: Jun. 1, 2023

(54) METHODS OF TREATING CONDITIONS CHARACTERIZED BY INSULIN DEFICIENCY IN ANIMALS

(71) Applicant: HOFFMAN TECHNOLOGIES LLC, New York, NY (US)

(72) Inventors: Steven HOFFMAN, Mahwah, NJ (US); John ROTHMAN, Lebanon, NJ (US)

(21) Appl. No.: 17/995,727

(22) PCT Filed: Apr. 8, 2021
(86) PCT No.: PCT/US2021/026370

§ 371 Date: Oct. 7, 2022

Related U.S. Application Data
(60) Provisional application No. 63/006,956, filed on Apr. 8, 2020.

(51) Int. Cl. A61K 38/28; A61K 9/00; A61K 47/26; A61K 47/18; A61K 47/10; A61K 47/14; A61K 47/22; A61K 47/12

(52) U.S. Cl. CPC A61K 38/28; A61K 9/0014; A61K 47/26; A61K 47/186; A61K 47/10; A61K 47/18; A61K 47/14; A61K 47/22; A61K 47/12

(57) ABSTRACT

The present disclosure is directed to methods of treating conditions characterized by insulin deficiency in animals by trans dermal insulin administration.

### CROSS-REFERENCE TO RELATED APPLICATION

This application claims the benefit of priority to U.S. Provisional Patent Application No. 63/006,956, filed on Apr. 8, 2020, the entirety of which is incorporated by reference herein.

### BACKGROUND

Animals such as household pets and farm animals may suffer from conditions characterized by insulin deficiency, including diabetes mellitus, hyperglycemia, and ketosis. These conditions are often treated by providing the animal insulin injections, sometimes multiple times per day. Administration of such injections burdens the animal's caretaker in a number of ways. For example, animals are often uncooperative and actively resist the injection. Moreover, if the caretaker misses a scheduled injection the animal can become hypoglycemic and experience other serious health conditions that necessitate expensive specialized care. See, e.g., Behrend, E. et al., 2018 AAHA Diabetes Management Guidelines for Dogs and Cats, J Am Anim Hosp Assoc 2018; 54:1-21. Thus, there exists a need for methods of treating animals by administering insulin in a manner that avoids injection.

### SUMMARY

The present disclosure is directed to methods of treating a condition characterized by insulin deficiency in an animal, comprising applying an insulin-containing composition to the skin or a mucous membrane of the animal for a time sufficient to achieve permeation of at least a portion of the insulin through the skin or the mucous membrane of the animal. In certain embodiments, the insulin-containing composition comprises a first component, a second component, a C₂₋₁₀alkyl alcohol, an organic acid having 1 to 25 carbon atoms, and insulin, wherein the first and second components are further defined herein.

### DETAILED DESCRIPTION OF ILLUSTRATIVE EMBODIMENTS

The present disclosure may be understood more readily by reference to the following detailed description of desired embodiments and the examples included therein. In the following specification and the claims that follow, reference will be made to a number of terms which have the following meanings.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, “alkyl” refers to straight chain and branched chains having the indicated number of carbon atoms, usually from 1 to 20 carbon atoms, for example 1 to 8 carbon atoms, such as 1 to 6 or 1 to 7 carbon atoms. For example C₁₋₆ alkyl encompasses both straight and branched chain alkyl of from 1 to 6 carbon atoms. When an alkyl residue having a specific number of carbons is named, all branched and straight chain versions having that number of carbons are intended to be encompassed; thus, for example, “butyl” is meant to include n-butyl, sec-butyl, isobutyl and t-butyl; “propyl” includes n-propyl and isopropyl. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like.

As used herein, “alkenyl” refers to an unsaturated branched or straight-chain alkyl group having at least one carbon-carbon double bond. The group may be in either the cis or trans configuration about the double bond(s). The group may also be an aromatic group, for example, a phenyl or phenylene moiety. Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; phenylene, and the like. In certain embodiments, an alkenyl group has from 2 to 20 carbon atoms.

As used herein, “alkynyl” refers to an unsaturated branched or straight-chain alkyl group having at least one carbon-carbon triple bond derived by the removal of two molecules of hydrogen from adjacent carbon atoms of the parent alkyl. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms.

The present disclosure is directed to methods of treating a condition characterized by insulin deficiency in an animal, comprising applying an insulin-containing composition to the skin or a mucous membrane of the animal for a time sufficient to achieve permeation of at least a portion of the insulin through the skin or the mucous membrane of the animal.

As used herein, “a condition characterized by insulin deficiency,” is any adverse health condition that results form a lack of insulin or insufficient insulin in the animal's bloodstream.

In some embodiments, the condition characterized by insulin deficiency is diabetes mellitus, hyperglycemia, or ketosis.

In some embodiments, the condition characterized by insulin deficiency is diabetes mellitus.

In other embodiments, the condition characterized by insulin deficiency is hyperglycemia.

In other embodiments, the condition characterized by insulin deficiency is ketosis.

The methods of the disclosure are performed on animals. As used herein, “animal” refers to any animal other than a human being. In some embodiments, the animal is a mammal.

In some embodiments, the animal is a domesticated animal, such as a dog or a cat.

In other embodiments, the animal is a farm animal, such as a cow, goat, sheep, pig, or horse.

In some embodiments, the animal is a dog, cat, rodent (such as a mouse, hamster, guniea pig, ferret), cow, goat, sheep, pig, or horse.

In some embodiments, the animal is a dog.

In other embodiments, the animal is a cat.

In some embodiments, the animal is a cow.

In some embodiments, the animal is a goat.

In the methods of the disclosure, the animal is administered an insulin-containing composition. As used herein, “insulin” refers to any type of insulin or insulin derivative suitable for administration to the animal. Examples of insulin that may be used in the compositions of the disclosure include bovine insulin, porcine insulin, human recombinant inulin, glargine, protamine zinc insulin, porcine insulin zinc, neutral recombinant human insulin, isophane, vetsulin insulin, lente insulin, and ultralente insulin. The insulin used in the composition will depend in part on the species of animal to be treated. Types of insulin suitable for treatment of specific animals are known to those skilled in the art.

As used herein, the terms “permeation” or “transdermal permeation” includes both percutaneous delivery and transmucosal delivery, that is, passage through skin or mucosal tissue and into the bloodstream. As used herein in reference to transdermal permeation, the term “enhancing” refers to increasing the rate at which a therapeutic agent penetrates the skin or mucosal tissue and enters the bloodstream.

In addition to containing insulin, the compositions used in the methods of the disclosure include a first component, a second component, an alcohol, an organic acid, and, optionally, water. In other embodiments, the insulin-containing compositions further comprise a second therapeutic agent.

According to the disclosure, the insulin-containing compositions include a first component which comprises
a compound of formula I

R—(OCH₂CH₂)_{y}—OH (I)
wherein R is C₁₋₂₀alkyl, C₂₋₂₀alkenyl; or C₂₋₂₀alkynyl; and y is 1 to 25;
a tetrafunctional block copolymer surfactant terminating in primary hydroxyl groups;
a sorbitan derivative;
a C₈₋₁₀alkyl ammonium salt;
a compound of formula II

HO—(CH₂CH₂P)ₘ—C(CH₃)(C₄H₉)—C═C—C(CH₃)(C₄H₉)—(OCH₂CH₂)ₙ—OH (II)
wherein m and n are each independently 1 to 25;
or a combination thereof.

In preferred embodiments of the disclosure, the first component is a compound of formula I. In some embodiments, R is C₁₋₂₀alkyl, which can either be a straight chain or branched alkyl. Preferred compounds of formula I wherein R is C₁₋₂₀alkyl include, for example, is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether (TRITON N-101), nonaethylene glycol monododecyl ether, 23-{[4-(2,4,4-trimethyl-2-pentanyl)cyclohexyl]oxy}-3,6,9,12,15,18,21-heptaoxatricosan-1-ol, and combinations thereof. Nonaethylene glycol monododecyl ether is particularly preferred.

In other embodiments, R is C₂₋₂₀alkenyl, which can either be a straight chain or branched alkenyl. Preferred compounds of formula I wherein R is C₂₋₂₀alkenyl include, for example, polyoxyl(10)oleyl ether, polyethylene glycol tert-octylphenyl ether (TRITON X-100), and combinations thereof.

In yet other embodiment, R is C₂₋₂₀alkynyl, which can either be a straight chain or branch alkynyl.

In those embodiments wherein the first component is a compound of formula I, y is 1 to 25. In preferred embodiments, y is 5 to 15, preferably 8 to 10, with 9 being particularly preferred. In other embodiments, y is 1, 2, 3, 4, 5, 6, 7, 8, 9, 10, 11, 12, 13, 14, 15, 16, 17, 18, 19, 20, 21, 22, 23, 24, or 25.

In other aspects of the disclosure, the first component is a tetrafunctional block copolymer surfactant terminating in primary hydroxyl groups. Such compounds are commercially available under the tradename TETRONIC and include ethylenediaminetetrakis(ethoxylate-Block-propoxylate).

In other embodiments of the disclosure, the first component is a sorbitan derivative, for example, polyoxyethylene sorbitan tetraoleate, 1,4-anhydro-6-O-palmitoyl-D-glucitol (sorbitan, monohexadecanoate), a polyethylene glycol sorbitan monolaurate (e.g., TWEEN 20, TWEEN 40, TWEEN 60, TWEEN 85), and combinations thereof.

In still other embodiments of the disclosure, the first component is a C₈₋₁₀alkyl ammonium salt, for example, methyltrialkyl(C₈-C₁₀)ammonium chloride (ADOGEN 464).

In other embodiments, the first component is a compound of formula II.

The compositions of the disclosure can comprise from about 0.1 vol. % to about 40 vol. % of the first component. In preferred embodiments, the compositions comprise from about 1 vol. % to about 40 vol. % of the first component. In other embodiments, the compositions comprise from about 0.1 vol. % to about 5 vol. % of the first component. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 vol. % of the first component.

According to the disclosure, the insulin-containing compositions include a second component that comprises
an compound of the formula III

R²—N(R¹)—C(O)—R³ (III)
wherein
each R¹ is independently H or C₁₋₃alkyl; and
R² and R³ are independently C₁₋₇alkyl or together with the atoms to which they are attached, form a lactam having 3 to 10 carbon atoms,
a sulfoxide;
a urea;
ethyl acetate;
or a combination thereof.

In preferred embodiments, the second component is compound of formula III. In some embodiments, R¹ is H. In other embodiments, R¹ is methyl, ethyl, propyl, or isopropyl, with methyl being particularly preferred.

In those embodiments wherein R² and R³ are independently C₁₋₇alkyl, each of R² and R³ is independently methyl, ethyl, propyl, isopropyl, butyl, s-butyl, t-butyl, pentyl, hexyl, or heptyl.

Preferably, R² and R³, together with the atoms to which they are attached, form a lactam having 3 to 10 carbon atoms. For example, the lactam can include 3, 4, 5, 6, 7, 8, 9, or 10 carbons, which can be a part of the lactam ring or which can form exocyclic branching. Examples of preferred lactams include pyrrolidones such as 2-pyrrolidone, 1-methyl-2-pyrrolidone, 5-methyl-2-pyrrolidone, and 1-ethyl-2-pyrrolidone. Preferably, the lactam is 1-methyl-2-pyrrolidinone or 2-pyrrolidone.

In some embodiments, the second component is a sulfoxide, for example, dimethyl sulfoxide.

In other embodiments, the second component is a urea, for example an imidazolidinone.

The compositions of the disclosure can comprise from about 0.01 vol. % to about 10 vol. % of the second component. In preferred embodiments, the compositions comprise from about 0.01 vol. % to about 5 vol. % of the second component. In other embodiments, the compositions comprise from about 0.01 vol. % to about 4 vol. % of the second component. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or about 10 vol. % of the second component.

In some embodiments of the disclosure, the ratio, by volume, of the first component to the second component is about 10:1 to about 4:1.

The insulin-containing compositions of the disclosure also include a C₂₋₁₀alkyl alcohol having at least one —OH moiety or at least two —OH moieties. For example, preferred alcohols include glycerol, propylene glycol, ethanol, isopropanol, 1-propanol, butanol, t-butanol, pentanol, 1-octanol, and combinations thereof, with ethanol being particularly preferred.

The compositions of the disclosure can comprise from about 0.1 vol. % to about 50 vol. % of the C₂₋₁₀ alkyl alcohol. In preferred embodiments, the compositions comprise from about 1 vol. % to about 50 vol. % of the C₂₋₁₀ alkyl alcohol. In other embodiments, the compositions comprise from about 0.1 vol. % to about 5 vol. % of the C₂₋₁₀ alkyl alcohol. For example, the compositions can comprise about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 vol. % of the C₂₋₁₀ alkyl alcohol.

The insulin-containing compositions of the disclosure also include an organic acid having 1 to 25 carbon atoms. For example, organic acids for use in the disclose compositions include acetic acid, ascorbic acid, lactic acid, glycolic acid, propionic acid, and combinations thereof.

Other organic acids for use in the disclosure include fatty acids. As used herein, the term “fatty acid” has its ordinary meaning as would be understood by a person of ordinary skill in the art and includes a molecule having a carboxylic group and a hydrocarbon chain. Descriptions of the number of carbon atoms in a fatty acid herein refer to the number of carbon atoms in the hydrocarbon chain of the fatty acid, irrespective of whether the hydrocarbon chain is straight or branched.

As used herein, the term “fatty acid” includes saturated fatty acids, which do not contain any double or triple bonds in the hydrocarbon chain. Saturated fatty acids include, but are not limited to propionic acid (C3) (by way of example, C3 indicates propionic acid has 3 carbon atoms in its hydrocarbon chain; the number of carbon atoms in the hydrocarbon chain of other example fatty acids is denoted in analogous fashion herein), butyric acid (C4), valeric acid (C5), caproic acid (C6), enanthic acid (C7), caprylic acid (C8), pelargonic acid (C9), capric acid (C10), undecylic acid (C11), lauric acid (C12), tridecylic acid (C13), myristic acid (C14), pentadecylic acid (C15), palmitic acid (C16), margaric acid (C17), stearic acid (C18), isostearic acid (C18), nonadecylic acid (C19), arachidic acid (C20), heneicosylic acid (C21), behenic acid (C22), tricosylic acid (C23), lignoceric acid (C24), pentacosylic acid (C25), cerotic acid (C26), heptacosylic acid (C27), montanic acid (C28), nonacocylic acid (C29), melissic acid (C30), henatriacontylic acid (C31), lacceroic acid (C32), psyllic acid (C33), geddic acid (C34), ceroplastic acid (C35) and hexatriacontylic acid (C36).

As used herein, the term “fatty acid” also includes monounsaturated fatty acids, which contain one double or triple bond in the hydrocarbon chain, and polyunsaturated fatty acids, which contain more than one double and/or triple bond in the hydrocarbon chain. Such acids include, but are not limited to the omega 3, omega 6, omega 9 fatty acids, other fatty acids such as myristoleic and palmitoleic acid and conjugated fatty acids. Examples of monounsaturated and polyunsaturated fatty acids include but are not limited to, (a) omega 3 fatty acids, such as hexadecatrienoic acid (C16:3); (by way of example, C16:3 indicates hexadecatrienoic acid has 16 carbon atoms in its hydrocarbon chain and 3 double bonds; the number of carbon atoms and double bonds in the hydrocarbon chain of other example unsaturated fatty acids is denoted in analogous fashion herein), alpha linolenic acid (C18:3) and eicosapentanoic acid (20:5), (b) omega 6 fatty acids, such as linoleic acid (18:2), docosadienoic acid (C22:2), arachidonic acid (C20:4) and tetracosatetraenoic acid (C24:5), (c) omega 9 fatty acids, such as oleic acid (C18:1), eicosenoic acid (C20:1) and nevronic acid (C24:1), and (d) conjugated fatty acids such as rumenic acid (C18:2), eleostatic acid (C18:3), and rumelenic acid (C18:3).

As used herein, the term “fatty acid” also includes branched fatty acids. Examples of branched fatty acids include, but are not limited to, monomethyl branched fatty acids, such as 14-methyl pentadecanoic acid, 6-methyl caprylic acid, 4-methyl-3-pentenoic acid, (pyroterebic acid), 2-methyl-2E-butenoic acid (tiglic acid), 2-methyl-2Z-butenoic acid (angelic acid), multimethyl branched acids, isoprenoid fatty acids (vittatalactone, all-trans-retinoic acid), branched methoxy fatty acids and hydroxy and other fatty acids such as 2-hydroxyoctanoic acid and 4-oxopentanoic acid.

The compositions of the disclosure can comprise from about 0.01 vol. % to about 15 vol. % of the organic acid. In some embodiment, the compositions comprise from about 1 vol % to about 15 vol % of the organic acid. In preferred embodiments, the compositions comprise from about 0.01 vol. % to about 5 vol. % of the organic acid. In other embodiments, the compositions comprise from about 0.01 vol. % to about 3 vol. % of the organic acid. For example, the compositions can comprise about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 vol. % of the organic acid.

Compositions of the disclosure can be anhydrous. As used herein, “anhydrous” refers to compositions comprising less than 1 vol. % of water, preferably less than 0.05 vol. % or less than 0.025 vol. % of water. Methods of determining water content are known in the art.

Compositions of the disclosure can include water. In some embodiments, the compositions can comprise up to 99 vol. % of water. In still other aspects, the compositions can comprise 5, 10, 20, 30, 40, 50, 60, 70, 80, 90, 95, or 99 vol. % of water. In other embodiments, the compositions can comprise 90, 91, 92, 93, 94, 95, 96, 97, 98, or 99 vol. % of water.

Compositions of the disclosure that include water can optionally contain one or more physiologically acceptable salts. While not being bound by any particular theory, it is believed that controlling the amount of salt that is present allows one to control the depth to which the present composition penetrate skin, with the concentration of salt having a generally inverse relationship to the penetration depth. Salts for use in the compositions include, but are not limited to, sodium chloride, potassium chloride, and mixtures thereof. A preferred form of sodium chloride is bacteriostatic sodium chloride solution.

The compositions of the disclosure can also include a second therapeutic agent (i.e., a therapeutic agent in addition to insulin). As used herein, the term “therapeutic agent” refers to a compound that, upon administration to the animal in a therapeutically effective amount, provides a therapeutic benefit to the patient. A therapeutic agent may be referred to herein as a drug or biologic. Those skilled in the art will appreciate that the term “therapeutic agent” is not limited to drugs or biologics, or to materials that have received regulatory approval.

For example, such second therapeutic agents include, but are not limited to, antibiotics, anticonvulsants, antiparisitics, sedatives, tranquilizers, anxiolytics, antiemetics, emetics, ACE inhibitors, adrenergic antagonists, antidepressants, antibacterials, calcium channel blockers, bronchodilators, hormones, steroids and their synthetic analogs, analgesics, narcotics, H2 antagonists, apetite stimulants, fungicides, germicides, antimicrobials, histamine blockers, antihistamines, vasodilators, insecticides, anti-inflammatories, anthelmintics, antipruritics, dopamine receptor agonists, antiseizure medications, phosphodiesterase 3 inhibitors, anticoccidials, antidotes, anesthetics, NSAIDS, hypnotics, beta blockers, alpha blockers, neuromuscular blocking agents, diuretics, dermatologicals, and antimetabolites.

Specific examples of therapeutic agents suitable for use in compositions of the invention include acepromazine, alprazolam, altrenogest, amantadine, aminophylline, amitraz, amitriptyline, amlodipine, amoxicillin, puniatozol, apomorphine, atenolol, atipamezole, Baytril, benazepril, bethanechol, bupivacaine, buprenorphine, butorphanol, carprofen, cefovecin, cefpodoxime, cephalexin, ceftiofur, chloral hydrate/magnesium sulfate/pentobarbital, chloramphenicol, cimetidine, ciprofloxacin, clamoxyquine, clavamox, clavaseptin, clavulanic acid, clenbuterol, clindamycin, clomipramine, cyproheptadine, deracoxib, dexamethasone, diazepam, dichlorophene, diphenhydramine, doxycycline, enalapril, enrofloxacin, equine chorionic gonadotropin, fenbendazole, fipronil, flunixin meglumine, furosemide, gabapentin, gentamicin/betamethasone valerate/clotrimazole, glycopyrrolate, hydromorphone, hydroxyzine, isoxsuprine, ivermectin, ketamine, ketoprofen, levamisole, levetiracetam, levothyroxine, loxicom, lufenuron, marbofloxacin, maropitant, mavacoxib, medetomidine, meloxicam, metacam, methimazole, metoclopramide, metronidazole, milbemycin oxime, mirtazapine, mitratapide, morphine, moxifloxacin, neomycin, nimesulide, nitarsone, nitenpyram, nitroscanate, nitroxynil, nystatin, oxytetracycline, ofloxacin, oclacitinib, omeprazole, oxibendazole, oxymorphone, pentoxyfylline, pergolide, phenylbutazone, phenylpropanolamine, pimobendan, pirlimycin, ponazuril, praziquantel, prazosin, prednisolone, prednisone, propofol, pyrantel, rafoxanide, rifampin, robenacoxib, roxarsone, selamectin, silver sulfadiazine, streptomycin, sucralfate, sulfasalazine, Telazol, tepoxalin, theophylline, thiostrepton, thiabendazole, tolfenamic acid, tramadol, triamcinolone acetonide, trimethoprim, trimethoprim/sulfadoxine, trilostane, tylosin, ursodeoxycholic acid (INN) or ursodiol (USAN), xylazine, and yohimbine.

The insulin-containing compositions of the invention may be formulated to be administered to the skin or mucosal tissue of the animal as gels, transdermal patches, lotions, creams, sprays, mists, emulsions, or dispersions. Appropriate excipients for formulating a gel, transdermal patch, lotion, cream, spray, or mist are readily apparent to a person of skill in the art and include, but are not limited to, stabilizers, emulsifiers, thickeners, antimicrobials, humectants, propellants, spreading agents, polymers, and adhesives, such as pressure sensitive adhesives. In particular, excipients that may be used to form a transdermal gel include, but are not limited to, alcohols, glycols, glycerin, butylated hydroxytoluene (BHT), and water.

In the methods of the disclosure, the insulin-containing composition is applied to the skin of the animal for a time sufficient to achieve permeation of at least a portion of the insulin through the skin or the mucous membrane of the animal. The compositions of the invention can, for example, be applied inside an animal's ear or to some other area of exposed skin. To the extent that application to an area of hair- or fur-bearing skin is desired, that area preferably is shaved or otherwise treated beforehand to remove at least some of the hair or fur. Skin permeation can be measured using techniques known in the art.

In those embodiments of the methods of the invention in which the insulin-containing composition contains a second therapeutic agent, the compositions are applied to the skin of the animal for a time sufficient to achieve permeation of at least a portion of the second therapeutic agent through the skin or the mucous membrane of the animal. Therapeutic agent skin permeation can be measured using techniques known in the art.

The compositions described herein can be applied to any convenient skin surface. Skin surfaces of interest include, but are not limited to: arms, leg, torso, head, neck, etc. In some embodiments, the animal's fur may be shaved from the skin surface to facilitate contact of the composition with the skin. The skin surface area that is covered by the transdermal formulation following application is generally sufficient to provide for the desired amount of insulin permeation, and in certain embodiments ranges from about 1 cm² to about 200 cm².

The compositions described herein can be applied a single time or a plurality of times over a given time period, e.g., the course of the disease condition being treated, where the dosing schedule when a plurality of applications over a given time period may be daily, weekly, biweekly, monthly, etc.

The compositions of the disclosure will, in some embodiments, include, in addition to the above-discussed components, one or more additional components. Additional components include, but are not limited to, a transdermal absorption enhancer, a preservative (e.g., paraben), an antioxidant, a stabilizing agent, a filling agent that contains a hydrophilic polymer; a cross-linking agents; and a plasticizing agent.

The following examples are provided to illustrate the methods, compositions, processes, and properties of the present disclosure. These examples are merely illustrative and not intended to limit the disclosure to the materials, conditions, or process parameters set forth therein.

### EXAMPLES

### Example 1. Aqueous, Insulin Transdermal Composition

Nonaethylene glycol monododecyl ether (3 mL), 1-methyl-2-pyrrolidinone (0.3 mL), ethanol (4 mL), and linoleic acid (1 mL) are combined. Insulin (3 mL, 100 units/mL, LANTUS SOLOSTAR, Sanofi) is then added to form an admixture. The admixture (1 mL) is then combined with 24 mL of water. The resulting aqueous composition can be applied to an animal's skin or mucus membrane for a time sufficient for the insulin to permeate through the skin or mucus membrane and into the animal's bloodstream to achieve a therapeutic effect.

### Example 2. Aqueous, Insulin Transdermal Composition

Nonaethylene glycol monododecyl ether (3 mL), 1-methyl-2-pyrrolidinone (0.3 mL), ethanol (4 mL), and linoleic acid (1 mL) are combined. Insulin (3 mL, 100 units/mL, LANTUS SOLOSTAR, Sanofi) is then added to form an admixture. The admixture (1 mL) is then combined with 32.3 mL of water. The resulting aqueous composition can be applied to an animal's skin or mucus membrane for a time sufficient for the insulin to permeate through the skin or mucus membrane and into the animal's bloodstream to achieve a therapeutic effect.

1. A method of treating a condition characterized by insulin deficiency in an animal, comprising applying an insulin-containing composition to the skin or a mucous membrane of the animal for a time sufficient to achieve permeation of at least a portion of the insulin through the skin or the mucous membrane of the animal,
wherein the composition comprises:
a first component comprising:
a compound of formula I
R—(OCH₂CH₂)_{y}—OH (I)
wherein R is C₁₋₂₀alkyl, C₂₋₂₀alkenyl; or C₂₋₂₀alkynyl; and y is 1 to 25;
a tetrafunctional block copolymer surfactant terminating in primary hydroxyl groups;
a sorbitan derivative;
a C₈₋₁₀alkyl ammonium salt;
a compound of formula II
HO—(CH₂CH₂O)ₘ—C(CH₃)(C₄H₉)—C≡C—C(CH₃)(C₄H₉)—(OCH₂CH₂)ₙ—OH (II)
wherein m and n are each independently 1 to 25;
or a combination thereof;
a second component comprising:
an amide of the formula III
R²—N(R¹)—C(O)—R³ (III)
wherein
each R¹ is independently H or C₁₋₃alkyl; and
R² and R³ are independently C₁₋₇alkyl or together with the atoms to which they are attached, form a lactam having 3 to 10 carbon atoms;
a sulfoxide;
a urea;
ethyl acetate;
or a combination thereof;
a C₂₋₁₀ alkyl alcohol;
an organic acid having 1 to 25 carbon atoms;
optionally, water; and
insulin.

2. The method of claim 1, wherein the condition characterized by insulin deficiency is diabetes mellitus, hyperglycemia, or ketosis.

3. (canceled)

4. (canceled)

5. (canceled)

6. The method of claim 1, wherein the animal is domesticated animal.

7. (canceled)

8. (canceled)

9. The method of claim 1, wherein the animal is a farm animal.

10. The method of claim 9, wherein the farm animal is a cow, sheep, goat, pig, or horse.

11. (canceled)

12. The method of claim 1, wherein the composition further comprises a physiologically acceptable salt.

13. The method of claim 12, wherein the physiologically acceptable salt is sodium chloride, potassium chloride, or a mixture thereof.

14. The method of claim 1, wherein the first component is the compound of formula I.

15. (canceled)

16. (canceled)

17. The method of claim 14, wherein the compound of formula I is cetomacrogol 1000; octadecan-1-ol, ethoxylated; polyoxyethylene(12)tridecyl ether; polyoxyethylene(10)tridecyl ether; fatty alcohol polyoxyethylene ether, polyoxyethylene branched nonylcyclohexyl ether, nonaethylene glycol monododecyl ether, 23-{[4-(2,4,4-trimethyl-2-pentanyl)cyclohexyl]oxy}-3,6,9,12,15,18,21-heptaoxatricosan-1-ol, or a combination thereof.

18. The method of claim 14, wherein the compound of formula I is nonaethylene glycol monododecyl ether.

19. (canceled)

20. The method of claim 1, wherein the compound of formula I is polyoxyl(10)oleyl ether, polyethylene glycol tert-octylphenyl ether, or a combination thereof.

21. (canceled)

22. The method claim 1, wherein the first component is a tetrafunctional block copolymer surfactant terminating in primary hydroxyl groups.

23. The method of claim 22, wherein the tetrafunctional block copolymer surfactant terminating in primary hydroxyl groups is ethylenediaminetetrakis(ethoxylate-Block-propoxylate).

24. The method of claim 1, wherein the first component is a sorbitan derivative.

25. The method of claim 24, wherein the sorbitan derivative is polyoxyethylene sorbitan tetraoleate, 1,4-anhydro-6-O-palmitoyl-D-glucitol (sorbitan, monohexadecanoate), a polyethylene glycol sorbitan monolaurate, or a combination thereof.

26. The method of claim 1, wherein the first component is a C₈₋₁₀alkyl ammonium salt.

27. The method of claim 26, wherein the C₈₋₁₀alkyl ammonium salt is methyltrialkyl(C₈-C₁₀)ammonium chloride (ADOGEN 464).

28. The method of claim 1, wherein the first component is the compound of formula II.

29. The method of claim 1, wherein the second component is the compound of formula III.

30. The method of claim 29, wherein R¹ is methyl, ethyl, or propyl.

31. The method of claim 29, wherein R² and R³, together with the atoms to which they are attached, form a lactam having 3 to 10 carbon atoms.

32. The method of claim 31, wherein the lactam is a pyrrolidone.

33. The method of claim 32, wherein the pyrrolidone is 1-methyl-2-pyrrolidinone.

34. The method of claim 1, wherein the second component is a sulfoxide.

35. The method of claim 1, wherein the second component is a urea.

36. The method of claim 1, wherein the second component is ethyl acetate.

37. The method of claim 1, wherein the C₂₋₁₀alkyl alcohol is glycerol, propylene glycol, ethanol, isopropanol, 1-propanol, butanol, t-butanol, pentanol, 1-octanol, or a combination thereof.

38. The method of claim 1, wherein the organic acid is a fatty acid or a C₁₋₆alkyl acid.

39. The method of claim 38, wherein the fatty acid is linoleic acid.

40. The method of claim 1, wherein the first component is nonaethylene glycol monododecyl ether; the second component is 1-methyl-2-pyrrolidinone; and the organic acid is linoleic acid.

41. The method of claim 1, wherein the composition is anhydrous.

42. The method of claim 1, wherein the composition further comprising water.

43. The method of method of claim 1, wherein the ratio (v/v) of the nonaethylene glycol monododecyl ether to the 1-methyl-2-pyrrolidone is from 9:1 to 11:1.

44. (canceled)

45. The method of claim 1, wherein the composition is formulated in the form of a gel, transdermal patch, lotion, cream, spray, emulsion, or dispersion.

46. The method of claim 1, wherein the composition further comprises a second therapeutic agent.
